(19) United States
(12) Patent Application Publication
(10) Pub. No.: US 2003/0200569 A1
(43) Pub. Date: Oct. 23, 2003

(54) Amino acid:glyoxylate aminotransferase genes from plants and uses thereof

(76) Inventors: Hong G. Nam, Pohang (KR); Ji H. Jun, Pohang (KR); Cheol Soo Kim, Tucson, AZ (US); Joshua Z. Levin, Raleigh, NC (US); Michael W. Bauer, Holly Springs, NC (US)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 09/837,534

(22) Filed: Apr. 18, 2001

Related U.S. Application Data
(60) Provisional application No. 60/325,764, filed on Apr. 18, 2000.

(51) Int. Cl. C12N 15/82; A01H 5/00; C12N 15/29; C12P 21/02

(52) U.S. Cl. 800/300; 800/278; 536/23.6; 530/370

(57) ABSTRACT

The present invention provides plant amino acid:glyoxylate aminotransferase genes. Also disclosed are the recombinant production of these plant amino acid:glyoxylate aminotransferase enzymes in heterologous hosts, screening chemicals for herbicidal activity using these recombinantly produced enzymes, and the use of thereby identified herbicidal chemicals to suppress the growth of undesired vegetation. Furthermore, the present invention provides methods for the development of herbicide tolerance in plants, plant tissues, plant seeds, and plant cells using the amino acid:glyoxylate aminotransferase genes of the invention.

### FIELD OF THE INVENTION

The invention relates generally to enzymatic activity involved in amino acid:glyoxylate aminotransferase in plants. In particular, the invention relates to plant genes that encode a polypeptide having amino acid:glyoxylate aminotransferase activity. The invention has various utilities, including the recombinant production of polypeptides having amino acid:glyoxylate aminotransferase activity in heterologous hosts, the screening of chemicals for herbicidal activity, and the use of thereby identified herbicidal chemicals to control the growth of undesired vegetation. The invention may also be applied to the development of herbicide tolerance in plants, plant tissues, plant seeds, and plant cells.

### BACKGROUND OF THE INVENTION

The use of herbicides to control undesirable vegetation such as weeds in crop fields has become almost a universal practice. The herbicide market exceeds 15 billion dollars annually. Despite this extensive use, weed control remains a significant and costly problem for farmers.

For example, present herbicides often impose special limitations on farming practices, and the time and method of application and stage of weed plant development often are critical for good weed control with such herbicides, thus creating farm management constraints. Furthermore, since only a few target enzymes are inhibited by currently used herbicides, various weed species are, or may become, resistant to these herbicides. For all of these reasons, the discovery and development of effective new herbicides, in particular those acting on novel target enzymes, is increasingly important.

Novel herbicides can now be discovered using high-throughput screens that implement recombinant DNA technology. Once identified, metabolic enzymes essential to plant growth and development can be recombinantly produced through standard molecular biological techniques and utilized as herbicide targets in screens for novel inhibitors of the enzyme's activity. The novel inhibitors discovered through such screens may then be used as herbicides to control undesirable vegetation. Such herbicides are also useful for selecting herbicide tolerant plants, and seed plants tolerant to the herbicide can be produced, for example by genetic engineering techniques. Thus, herbicides that exhibit greater potency, broader weed spectrum, and more rapid degradation in soil can be applied to crops that are resistant or tolerant to herbicides in order to kill weeds without attendant risk of damage to the crop.

Therefore, in order to meet the future food requirements of the world's growing population in a cost-effective and environmentally safe manner, there exists a long felt and unfulfilled need for novel target enzymes for herbicides, for new and better herbicides inhibiting such target enzymes and for plants tolerant to these new and better herbicides.

### SUMMARY OF THE INVENTION

In view of these long felt yet unfulfilled needs, one object of the invention is to provide a method for identifying new or improved herbicides. Another object of the invention is to provide a method for using such new or improved herbicides to suppress the growth of plants such as weeds. Still another object of the invention is to provide improved crop plants, and seed thereof, that are tolerant to such new or improved herbicides.

In furtherance of these and other objects, the present invention provides a DNA molecule comprising a nucleotide sequence, preferably isolated from a plant, that encodes a polypeptide having amino acid:glyoxylate aminotransferase activity. The inventors are the first to demonstrate that the amino acid:glyoxylate aminotransferase gene is essential for the growth of a plant, and is therefore a good target enzyme for identifying new herbicides. In particular, the present invention provides a DNA molecule isolated from Arabidopsis thaliana that encodes amino acid:glyoxylate aminotransferase, preferably a serine:glyoxylate aminotransferase or an alanine:glyoxylate aminotransferase, more preferably an alanine:glyoxylate aminotransferase, and DNA molecules substantially identical thereto that encode enzymes having amino acid:glyoxylate aminotransferase activity, preferably serine:glyoxylate aminotransferase or alanine:glyoxylate aminotransferase activity, more preferably alanine:glyoxylate activity. The nucleotide sequence of the present invention as well as the protein encoded by the present invention are referred to as amino acid:glyoxylate aminotransferase. According to one embodiment, the present invention provides a DNA molecule comprising a nucleotide sequence isolated from a plant that encodes the polypeptide set forth in SEQ ID NO:2. For example, the DNA molecule of the invention may comprise a nucleotide sequence set forth in SEQ ID NO:1. In another example, the DNA molecule of the invention comprises a nucleotide sequence that is substantially identical to the coding sequence set forth in SEQ ID NO:1 and that encodes a polypeptide having amino acid:glyoxylate aminotransferase activity, preferably serine:glyoxylate aminotransferase or alanine:glyoxylate aminotransferase activity, more preferably alanine:glyoxylate activity. Although the nucleotide sequence provided in SEQ ID NO:1 is isolated from Arabidopsis thaliana, using the information provided by the present invention, other nucleotide sequences that encode a polypeptide having amino acid:glyoxylate aminotransferase activity are obtained from other sources, e.g. from other plants, using standard methods known in the art.

The present invention also provides a nucleotide sequence construct comprising a promoter operatively linked to a DNA molecule of the invention. Further, the present invention provides methods to stably transform such a nucleotide sequence construct into a host cell, and host cells comprising such a nucleotide sequence construct, wherein the host cell is capable of expressing the DNA molecule encoding a polypeptide having amino acid:glyoxylate aminotransferase activity. Any suitable cell may be used as a host cell, e.g. a bacterial cell, a yeast cell, or a plant cell.

In accordance with another embodiment, the present invention also relates to the recombinant production of a amino acid:glyoxylate aminotransferase polypeptide and methods of use of amino acid:glyoxylate aminotransferase in assays for identifying compounds that interact with amino acid:glyoxylate aminotransferase polypeptide. In a preferred embodiment, the present invention provides a plant polypeptide having amino acid:glyoxylate aminotransferase activity useful for identifying inhibitors of amino acid:glyoxylate aminotransferase activity in in vivo and in vitro assays. Preferably the isolated polypeptide of the present invention comprises an amino acid sequence substantially identical to the amino acid sequence set forth in SEQ ID NO:2. More preferably, this enzyme comprises the amino acid sequence set forth in SEQ ID NO:2.

The present invention further provides methods of using purified polypeptides having amino acid:glyoxylate aminotransferase activity, preferably polypeptides derived from plant sources, in assays to screen for and identify compounds that interact with a amino acid:glyoxylate aminotransferase polypeptide. Such compounds are preferably inhibitors of amino acid:glyoxylate aminotransferase activity, and are potentially herbicides of future commercial interest. The inhibitors are used as herbicides to suppress the growth of undesirable vegetation in fields where crops are grown, particularly agronomically important crops such as maize and other cereal crops such as wheat, oats, rye, sorghum, rice, barley, millet, turf and forage grasses, and the like, as well as cotton, sugar cane, sugar beet, oilseed rape, and soybeans.

Thus, an assay useful for identifying inhibitors of essential plant genes, such as plant amino acid:glyoxylate aminotransferase genes, comprises the steps of:

a) reacting a plant amino acid:glyoxylate aminotransferase enzyme and a substrate thereof in the presence of a suspected inhibitor of the enzyme's function;

b) comparing the rate of enzymatic activity in the presence of the suspected inhibitor to the rate of enzymatic activity under the same conditions in the absence of the suspected inhibitor; and

c) determining whether the suspected inhibitor inhibits the amino acid:glyoxylate aminotransferase enzyme.

For example, the inhibitory effect on plant amino acid:glyoxylate aminotransferase may be determined by a reduction or complete inhibition of amino acid:glyoxylate aminotransferase activity in the assay. Such a determination may be made by comparing, in the presence and absence of the candidate inhibitor, the amount of substrate used or intermediate or product made during the reaction.

The present invention further embodies plants, plant tissues, plant seeds, and plant cells that have modified amino acid:glyoxylate aminotransferase activity and that are therefore tolerant to inhibition by a chemical at levels normally inhibitory to naturally occurring amino acid:glyoxylate aminotransferase enzyme activity. Herbicide tolerant plants encompassed by the invention include those that would otherwise be potential targets for normally inhibiting herbicides, particularly the agronomically important crops mentioned above. According to one aspect of this embodiment, plants, plant tissue, plant seeds, or plant cells are stably transformed with a recombinant DNA molecule comprising a suitable promoter functional in plants operatively linked to a nucleotide sequence that encodes an enzyme having modified amino acid:glyoxylate aminotransferase activity that is tolerant to a concentration of a amino acid:glyoxylate aminotransferase inhibitor that would normally inhibit the activity of wild-type, unmodified amino acid:glyoxylate aminotransferase in the plant. Modified amino acid:glyoxylate aminotransferase activity may also be conferred upon a plant by increasing expression of wild-type (i.e. sensitive) amino acid:glyoxylate aminotransferase enzyme by providing multiple copies of wild-type amino acid:glyoxylate aminotransferase genes to the plant or by overexpression of the endogenous wild-type amino acid:glyoxylate aminotransferase gene, or genes, under control of a stronger-than-wild-type promoter (e.g. either a promoter that drives expression at a higher rate, or a promoter that drives expression for a longer duration). The transgenic plants, plant tissue, plant seeds, or plant cells thus created are then selected by conventional selection techniques, whereby inhibitor tolerant descendants (lines) are isolated, characterized, and developed. Alternately, random or site-specific mutagenesis may be used to generate amino acid:glyoxylate aminotransferase inhibitor tolerant lines. Still further, inhibitor tolerant lines can be developed via selection of natural variants.

Therefore, the present invention provides a plant, plant cell, plant seed, or plant tissue comprising a DNA molecule comprising a nucleotide sequence, preferably isolated from a plant, that encodes an enzyme having amino acid:glyoxylate aminotransferase and wherein the DNA molecule confers upon the plant, plant cell, plant seed, or plant tissue tolerance to a amino acid:glyoxylate aminotransferase inhibitor in amounts that normally naturally occurring amino acid:glyoxylate aminotransferase activity. According to one example of this embodiment, the enzyme comprises an amino acid sequence substantially identical to the amino acid sequence set forth in SEQ ID NO:2. According to another example of this embodiment, the DNA molecule is substantially identical to the coding sequence set forth in SEQ ID NO:1. In a related aspect, the present invention is directed to a method for selectively suppressing the growth of weeds in a field containing a crop of planted crop seeds or plants, comprising applying to crops or crop seeds that are tolerant to an inhibitor that inhibits naturally occurring amino acid:glyoxylate aminotransferase activity and the weeds in the field an amino acid:glyoxylate aminotransferase inhibitor in amounts that inhibit naturally occurring amino acid:glyoxylate aminotransferase activity, wherein the inhibitor suppresses the growth of the weeds without significantly suppressing the growth of the crops.

Other objects and advantages of the present invention will become apparent to those skilled in the art from a study of the following description of the invention and non-limiting examples.

The invention thus provides:

An isolated DNA molecule comprising a nucleotide sequence substantially identical to SEQ ID NO:1. In a preferred embodiment, the nucleotide sequence encodes an amino acid sequence substantially identical to SEQ ID NO:2. In another preferred embodiment, the nucleotide sequence is SEQ ID NO:1. In yet another preferred embodiment, the nucleotide sequence encodes the amino acid sequence of SEQ ID NO:2. Preferably, the nucleotide sequence is a plant nucleotide sequence, which preferably encodes a polypeptide having amino acid:glyoxylate aminotransferase activity, preferably serine:glyoxylate aminotransferase or alanine:glyoxylate aminotransferase activity, more preferably alanine:glyoxylate activity.

The invention further provides:

A polypeptide comprising an amino acid sequence encoded by a nucleotide sequence substantially identical to SEQ ID NO:1. Preferably, the amino acid sequence is encoded by SEQ ID NO:1. Preferably, the polypeptide comprises an amino acid sequence substantially identical to SEQ ID NO:2. Preferably the amino acid sequence is SEQ ID NO:2. The amino acid sequence preferably has amino acid:glyoxylate aminotransferase activity, preferably serine:glyoxylate aminotransferase or alanine:glyoxylate aminotransferase activity, more preferably alanine:glyoxylate activity.

In another preferred embodiment, the amino acid sequence comprises at least 20 consecutive amino acid residues of the amino acid sequence encoded by SEQ ID NO:1. Or, alternatively, the amino acid sequence comprises at least 20 consecutive amino acid residues of the amino acid sequence of SEQ ID NO:2.

The invention further provides:

An expression cassette comprising a promoter operatively linked to a DNA molecule according to the present invention, wherein the promoter is preferably functional in a eukaryote, wherein the promoter is preferably heterologous to the DNA molecule. The present invention further provides recombinant vector comprising an expression cassette according to the present invention, wherein said vector is preferably capable of being stably transformed into a host cell, a host cell comprising a DNA molecule according to the present invention, wherein said DNA molecule is preferably expressible in the cell. The host cell is preferably selected from the group consisting of an insect cell, a yeast cell, a prokaryotic cell and a plant cell. The invention further provides a plant or seed comprising a plant cell of the present invention, wherein the plant or seed is preferably tolerant to an inhibitor of amino acid:glyoxylate aminotransferase activity, preferably serine:glyoxylate aminotransferase or alanine:glyoxylate aminotransferase activity, more preferably alanine:glyoxylate activity.

The invention further provides:

A process for making nucleotides sequences encoding gene products having altered amino acid:glyoxylate aminotransferase activity comprising: a) shuffling an unmodified nucleotide sequence of the present invention, b) expressing the resulting shuffled nucleotide sequences, and c) selecting for altered amino acid:glyoxylate aminotransferase activity as compared to the amino acid:glyoxylate aminotransferase activity of the gene product of said unmodified nucleotide sequence. The gene product has preferably altered serine:glyoxylate aminotransferase or alanine:glyoxylate aminotransferase activity, more preferably altered alanine:glyoxylate activity.

In a preferred embodiment, the unmodified nucleotide sequence is identical or substantially identical to SEQ ID NO:1, or a homolog thereof. The present invention further provides a DNA molecule comprising a shuffled nucleotide sequence obtainable by the process described above, a DNA molecule comprising a shuffled nucleotide sequence produced by the process described above. Preferably, a shuffled nucleotide sequence obtained by the process described above has enhanced tolerance to an inhibitor of amino acid:glyoxylate aminotransferase activity. The invention further provides an expression cassette comprising a promoter operatively linked to a DNA molecule comprising a shuffled nucleotide sequence a recombinant vector comprising such an expression cassette, wherein said vector is preferably capable of being stably transformed into a host cell, a host cell comprising such an expression cassette, wherein said nucleotide sequence is preferably expressible in said cell. A preferred host cell is selected from the group consisting of an insect cell, a yeast cell, a prokaryotic cell and a plant cell. The invention further provides a plant or seed comprising such plant cell, wherein the plant is preferably tolerant to an inhibitor of amino acid:glyoxylate aminotransferase activity.

The invention further provides:

A method for selecting compounds that interact with the protein encoded by SEQ ID NO:1, comprising: a) expressing a DNA molecule comprising SEQ ID NO:1 or a sequence substantially identical to SEQ ID NO:1, or a homolog thereof, to generate the corresponding protein, b) testing a compound suspected of having the ability to interact with the protein expressed in step (a), and c) selecting compounds that interact with the protein in step (b).

The invention further provides:

A process of identifying an inhibitor of amino acid:glyoxylate aminotransferase activity, preferably serine:glyoxylate aminotransferase or alanine:glyoxylate aminotransferase activity, more preferably alanine:glyoxylate activity, comprising: a) introducing a DNA molecule comprising a nucleotide sequence of SEQ ID NO:1 and having amino acid:glyoxylate aminotransferase activity, or nucleotide sequences substantially identical thereto, or a homolog thereof, into a plant cell, such that said sequence is functionally expressible at levels that are higher than wild-type expression levels, b) combining said plant cell with a compound to be tested for the ability to inhibit the amino acid:glyoxylate aminotransferase activity under conditions conducive to such inhibition, c) measuring plant cell growth under the conditions of step (b), d) comparing the growth of said plant cell with the growth of a plant cell having unaltered amino acid:glyoxylate aminotransferase activity under identical conditions, and e) selecting said compound that inhibits plant cell growth in step (d).

The invention further comprises a compound having herbicidal activity identifiable according to the process described immediately above.

The invention further comprises:

A process of identifying compounds having herbicidal activity comprising:

a) combining a protein of the present invention and a compound to be tested for the ability to interact with said protein, under conditions conducive to interaction, b) selecting a compound identified in step (a) that is capable of interacting with said protein, c) applying identified compound in step (b) to a plant to test for herbicidal activity, and d) selecting compounds having herbicidal activity.

The invention further comprises a compound having herbicidal activity identifiable according to the process described immediately above.

The invention further comprises:

A method for suppressing the growth of a plant comprising, applying to said plant a compound that inhibits the activity of a polypeptide of the present invention in an amount sufficient to suppress the growth of said plant.

The invention further comprises:

A method for recombinantly expressing a protein having amino acid:glyoxylate aminotransferase activity comprising introducing a nucleotide sequence encoding a protein having one of the above activities into a host cell and expressing the nucleotide sequence in the host cell. A preferred host cell is selected from the group consisting of an insect cell, a yeast cell, a prokaryotic cell and a plant cell. A preferred prokaryotic cell is a bacterial cell, e.g. E. coli.

### BRIEF DESCRIPTION OF THE SEQUENCES IN THE SEQUENCE LISTING

SEQ ID NO:1 is an mRNA sequence encoding amino acid:glyoxylate aminotransferase from Arabidopsis thaliana.

SEQ ID NO:2 is the predicted amino acid sequence of Arabidopsis thaliana amino acid:glyoxylate aminotransferase encoded by SEQ ID NO:1.

SEQ ID NO:3 is the 5′ portion of the partial cDNA sequence of the amino acid:glyoxylate aminotransferase gene isolated from the 35-191 line of Arabidopsis thaliana.

SEQ ID NO:4 is the 3′ portion of the partial cDNA sequence of the amino acid:glyoxylate aminotransferase gene isolated from the 35-191 line of Arabidopsis thaliana.

SEQ ID NO:5 is the oligonucleotide A

SEQ ID NO:6 is the oligonucleotide P1

SEQ ID NO:7 is the oligonucleotide P4

### DEFINITIONS

For clarity, certain terms used in the specification are defined and used as follows:

Activatable DNA Sequence: a DNA sequence that regulates the expression of genes in a genome, desirably the genome of a plant. The activatable DNA sequence is complementary to a target gene endogenous in the genome, in this case the gene encoding amino acid:glyoxylate aminotransferase. When the activatable DNA sequence is introduced and expressed in a cell, it inhibits expression of the target gene. An activatable DNA sequence useful in conjunction with the present invention includes those encoding or acting as dominant inhibitors, such as a translatable or untranslatable sense sequence capable of disrupting gene function in stably transformed plants to positively identify one or more genes essential for normal growth and development of a plant. A preferred activatable DNA sequence is an antisense DNA sequence. The interaction of the antisense sequence and the target gene results in substantial inhibition of the expression of the target gene so as to kill the plant, or at least inhibit normal plant growth or development.

Activatable DNA Construct: a recombinant DNA construct comprising a synthetic promoter operatively linked to the activatable DNA sequence, which when introduced into a cell, desirably a plant cell, is not expressed, i.e. is silent, unless a complete hybrid transcription factor capable of binding to and activating the synthetic promoter is present. The activatable DNA construct is introduced into cells, tissues, or plants to form stable transgenic lines capable of expressing the activatable DNA sequence.

Antiparallel: “Antiparallel” refers herein to two nucleotide sequences paired through hydrogen bonds between complementary base residues with phosphodiester bonds running in the 5′-3′ direction in one nucleotide sequence and in the 3′-5′ direction in the other nucleotide sequence.

Co-factor: natural reactant, such as an organic molecule or a metal ion, required in an enzyme-catalyzed reaction. A co-factor is e.g. NAD(P), riboflavin (including FAD and FMN), folate, molybdopterin, thiamin, biotin, lipoic acid, pantothenic acid and coenzyme A, S-adenosylmethionine, pyridoxal phosphate, ubiquinone, menaquinone. Optionally, a co-factor can be regenerated and reused.

Complementary: “Complementary” refers to two nucleotide sequences which comprise antiparallel nucleotide sequences capable of pairing with one another upon formation of hydrogen bonds between the complementary base residues in the antiparallel nucleotide sequences.

DNA shuffling: DNA shuffling is a method to rapidly, easily and efficiently introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA encodes an enzyme modified with respect to the enzyme encoded by the template DNA, and preferably has an altered biological activity with respect to the enzyme encoded by the template DNA.

Enzyme activity: means herein the ability of an enzyme to catalyze the conversion of a substrate into a product. A substrate for the enzyme comprises the natural substrate of the enzyme but also comprises analogues of the natural substrate, which can also be converted, by the enzyme into a product or into an analogue of a product. The activity of the enzyme is measured for example by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g. ATP, phosphoenolpyruvate, acetyl phosphate or phosphocreatine) remaining in the reaction mixture after a certain period of time or by determining the amount of a used donor of free energy or energy-rich molecule (e.g. ADP, pyruvate, acetate or creatine) in the reaction mixture after a certain period of time.

Essential: An “essential” gene is a gene encoding a protein such as e.g. a biosynthetic enzyme, receptor, signal transduction protein, structural gene product, or transport protein that is essential to the growth or survival of the plant.

Expression cassette: “Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus. In the case of a multicellular organism, such as a plant, the promoter can also be specific to a particular tissue or organ or stage of development. In the case of a plastid expression cassette, for expression of the nucleotide sequence from a plastid genome, additional elements, i.e. ribosome binding sites, may be required.

Herbicide: a chemical substance used to kill or suppress the growth of plants, plant cells, plant seeds, or plant tissues.

Heterologous DNA Sequence: a DNA sequence not naturally associated with a host cell into which it is introduced, including non-naturally occurring multiple copies of a naturally occurring DNA sequence.

Homologous DNA Sequence: a DNA sequence naturally associated with a host cell.

Inhibitor: a chemical substance that inactivates the enzymatic activity of amino acid:glyoxylate aminotransferase. The term “herbicide” is used herein to define an inhibitor when applied to plants, plant cells, plant seeds, or plant tissues.

Isogenic: plants which are genetically identical, except that they may differ by the presence or absence of a heterologous DNA sequence.

Isolated: in the context of the present invention, an isolated DNA molecule or an isolated enzyme is a DNA molecule or enzyme which, by the hand of man, exists apart from its native environment and is therefore not a product of nature. An isolated DNA molecule or enzyme may exist in a purified form or may exist in a non-native environment such as, for example, in a transgenic host cell.

Mature protein: protein which is normally targeted to a cellular organelle, such as a chloroplast, and from which the transit peptide has been removed.

Minimal Promoter: promoter elements, particularly a TATA element, that are inactive or that have greatly reduced promoter activity in the absence of upstream activation. In the presence of a suitable transcription factor, the minimal promoter functions to permit transcription.

Modified Enzyme Activity: enzyme activity different from that which naturally occurs in a plant (i.e. enzyme activity that occurs naturally in the absence of direct or indirect manipulation of such activity by man), which is tolerant to inhibitors that inhibit the naturally occurring enzyme activity.

Native: A “native” refers to a gene which is present in the genome of the untransformed plant cell.

Plant: A “plant” refers to any plant or part of a plant at any stage of development. Therein are also included cuttings, cell or tissue cultures and seeds. As used in conjunction with the present invention, the term “plant tissue” includes, but is not limited to, whole plants, plant cells, plant organs, plant seeds, protoplasts, callus, cell cultures, and any groups of plant cells organized into structural and/or functional units.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

In its broadest sense, the term “substantially identical”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence. Desirably, the substantially identical nucleotide sequence encodes the polypeptide encoded by the reference nucleotide sequence. The term “substantially identical” is specifically intended to include nucleotide sequences wherein the sequence has been modified to optimize expression in particular cells. Preferably, “substantially identical” refers to nucleotide sequences that encode a protein having at least 85% identity, preferably at least 90% identity, more preferably at least 95% identity, yet still more preferably at least 99% identity, to SEQ ID NO:2, wherein said protein sequence comparisons are conducted using GAP analysis as described below. Also, “substantially identical” preferably also refers to nucleotide sequences having at least 73% identity, more preferably 85% identity, more preferably at least 90% identity, still more preferably 95% identity, yet still more preferably at least 99% identity, to SEQ ID NO:1, wherein said nucleotide sequence comparisons are conducted using GAP analysis as described below. A nucleotide sequence “substantially identical” to the reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C. As used herein the term “amino acid:glyoxylate aminotransferase gene” refers to a DNA molecule comprising SEQ ID NO:1 or comprising a nucleotide sequence substantially identical to SEQ ID NO:1. Homologs of the amino acid:glyoxylate aminotransferase gene include nucleotide sequences that encode an amino acid sequence that is at least 28% identical to SEQ ID NO:2, more preferably at least 30%, still more preferably at least 35%, yet still more preferably at least 50%, still more preferably at least 85%, yet still more preferably at least 90%, as measured, using the parameters described below, wherein the amino acid sequence encoded by the homolog has the biological activity of the amino acid:glyoxylate aminotransferase protein.

The term “substantially identical”, when used herein with respect to a protein, means a protein corresponding to a reference protein, wherein the protein has substantially the same structure and function as the reference protein, e.g. where only changes in amino acids sequence not affecting the polypeptide function occur. When used for a protein or an amino acid sequence the percentage of identity between the substantially identical and the reference protein or amino acid sequence desirably is preferably at least 85%, more preferably 90%, more preferably at least 95%, still more preferably at least 99% using default GAP analysis parameters with the University of Wisconsin GCG (version 10), SEQWEB application of GAP, based on the algorithm of Needleman and Wunsch (Needleman and Wunsch (1970) J Mol. Biol. 48: 443-453). As used herein the term “amino acid:glyoxylate aminotransferase protein” refers to an amino acid sequence encoded by a DNA molecule comprising a nucleotide sequence substantially identical to SEQ ID NO:1. Homologs of the amino acid:glyoxylate aminotransferase protein are amino acid sequences that are at least 28% identical to SEQ ID NO:2, more preferably at least 30%, still more preferably at least 35%, yet still more preferably at least 50%, still more preferably at least 85%, yet still more preferably at least 90%, as measured using the parameters described above, wherein the amino acid sequence encoded by the homolog has the biological activity of the amino acid:glyoxylate aminotransferase protein.

Substrate: a substrate is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

Target gene: A “target gene” is any gene in a plant cell. For example, a target gene is a gene of known function or is a gene whose function is unknown, but whose total or partial nucleotide sequence is known. Alternatively, the function of a target gene and its nucleotide sequence are both unknown. A target gene is a native gene of the plant cell or is a heterologous gene which had previously been introduced into the plant cell or a parent cell of said plant cell, for example by genetic transformation. A heterologous target gene is stably integrated in the genome of the plant cell or is present in the plant cell as an extrachromosomal molecule, e.g. as an autonomously replicating extrachromosomal molecule.

Tolerance: the ability to continue essentially normal growth or function (i.e. no more than 5% of herbicide tolerant plants show phytotoxicity) when exposed to an inhibitor or herbicide in an amount sufficient to suppress the normal growth or function of native, unmodified plants.

Transformation: a process for introducing heterologous DNA into a cell, tissue, or plant. Transformed cells, tissues, or plants are understood to encompass not only the end product of a transformation process, but also transgenic progeny thereof.

Transgenic: stably transformed with a recombinant DNA molecule that preferably comprises a suitable promoter operatively linked to a DNA sequence of interest.

### DETAILED DESCRIPTION OF THE INVENTION

I. Plant Amino Acid:Glyoxylate Aminotransferase Genes

In one aspect, the present invention is directed to a DNA molecule comprising a nucleotide sequence isolated from a plant source that encodes amino acid:glyoxylate aminotransferase, preferably the nucleotide sequence encodes an alanine:glyoxylate aminotransferase or a serine:glyoxylate aminotransferase, more preferably the nucleotide sequence encodes an alanine:glyoxylate aminotransferase. In particular, the present invention provides a DNA molecule isolated from Arabidopsis thaliana that encodes amino acid:glyoxylate aminotransferase, preferably a serine:glyoxylate aminotransferase or an alanine:glyoxylate aminotransferase, more preferably an alanine:glyoxylate aminotransferase, and DNA molecules substantially identical thereto that encode enzymes having amino acid:glyoxylate aminotransferase activity, preferably serine:glyoxylate aminotransferase or alanine:glyoxylate aminotransferase activity, more preferably alanine:glyoxylate activity. The DNA coding sequence for amino acid:glyoxylate aminotransferase from Arabidopsis thaliana is provided in SEQ ID NO:1. The DNA sequence corresponding to the genomic sequence of the amino acid:glyoxylate aminotransferase gene from Arabidopsis thaliana is found in Genbank accession # AC007209.

There are several classes of amino acid:glyoxylate aminotransferases, for example, alanine:glyoxylate aminotransferase and serine:glyoxylate aminotransferase. A nucleotide sequence of the present invention preferably encodes a protein having amino acid: glyoxylate aminotransferase activity, preferably serine:glyoxylate aminotransferase or alanine:glyoxylate aminotransferase activity, more preferably alanine:glyoxylate activity. Such protein is referred to as amino acid: glyoxylate aminotransferase, preferably serine:glyoxylate aminotransferase or alanine:glyoxylate aminotransferase, more preferably alanine:glyoxylate.

Alanine:glyoxylate aminotransferase, also known as alanine—glyoxylate transaminase, (EC 2.6.1.44) is an enzyme catalyzing a biochemical reaction involved in amino acid metabolism and the glycolate pathway. This enzyme catalyzes the conversion of L-alanine and glyoxylate to pyruvate and glycine. Although the Arabidopsis thaliana protein from line 35-191, encoded by SEQ ID NO:2, is annotated as an alanine:glyoxylate aminotransferase, this protein has significant similarity to serine—glyoxylate transaminases, also known as serine—glyoxylate aminotransferases (E.C. 2.6.1.45). This enzyme catalyzes the conversion of L-serine and glyoxylate to 3-hydroxypyruvate and glycine as part of amino acid metabolism and the glycolate pathway. In addition, serine—glyoxylate aminotransferase is required in the photorespiratory pathway (Somerville (1984) Oxford Surv. of Plant. Mol. & Cell Biol., 1:103-131) and Arabidopsis thaliana mutants lacking this enzymatic activity are shown to have a conditional lethal phenotype (Somerville and Ogren, (1980) Proc. Natl. Acad. Sci. USA, 77:2684-2687). While this finding suggests that the serine—glyoxylate aminotransferase gene is essential for the growth of Arabidopsis thaliana, no DNA sequence information about the lethal mutants is published, and there is no indication or suggestion that alterations in the DNA sequence of SEQ ID NO:1 are the cause of the lethality observed in the serine:glyoxylate aminotransferase-lacking mutants. The work described in the present invention shows that the gene encoded by SEQ ID NO:1 is essential.

Proteins similar to that encoded by the nucleotide sequence isolated from the 35-191 line are identified based on DNA sequences from many organisms, including Arabidopsis thaliana (GenPept accession # AAC26854), Fritillaria agrestis (GenPept accession # AAB95218), Anabaena cylindrica (GenPept accession # CAA35518), Synechocystis sp. (GenPept accession # BAA18375), human (GenPept accession # AAA51680), and Saccharomyces cerevisiae (GenPept accession # BAA09208). Results from GAP analysis of the above sequences show the following identities at the amino acid level relative to Arabidopsis thaliana: Fritillaria agrestis (85.8% identical), Anabaena cylindrica (35.1% identical), Synechocystis sp (35.0% identical), human (30.8% identical), and Saccharomyces cerevisiae (28.6% identical), and the following identities at the nucleotide level relative to Arabidopsis thaliana: Fritillaria agrestis (73.5% identical).

Based on Applicants' disclosure of the present invention, amino acid:glyoxylate aminotransferase homologs, i.e. DNA sequences encoding amino acid:glyoxylate aminotransferase enzymes, are isolated from the genome of any desired plant. Alternatively, amino acid:glyoxylate aminotransferase gene sequences can be isolated from any plant according to well known techniques based on their sequence similarity to the Arabidopsis thaliana coding sequences (SEQ ID NO:1) taught by the present invention. In these techniques, all or part of a known amino acid:glyoxylate aminotransferase gene's coding sequence is used as a probe that selectively hybridizes to other amino acid:glyoxylate aminotransferase gene sequences present in a population of cloned genomic DNA fragments or cDNA fragments (i.e. genomic or cDNA libraries) from a chosen source organism. Such techniques include hybridization screening of plated DNA libraries (either plaques or colonies; see, e.g. Sambrook et al., “Molecular Cloning”, eds., Cold Spring Harbor Laboratory Press. (1989)) and amplification by PCR using oligonucleotide primers corresponding to sequence domains conserved among known amino acid:glyoxylate aminotransferase enzyme's amino acid sequences (see, e.g. Innis et al., “PCR Protocols, a Guide to Methods and Applications”, Academic Press (1990)). These methods are particularly well suited to the isolation of amino acid:glyoxylate aminotransferase gene sequences from organisms closely related to the organism from which the probe sequence is derived. The, application of these methods using the Arabidopsis coding sequences as probes is well suited for the isolation of amino acid:glyoxylate aminotransferase gene sequences from any source organism, preferably other plant species, including monocotyledons and dicotyledons.

The isolated amino acid:glyoxylate aminotransferase gene sequences taught by the present invention the manipulated according to standard genetic engineering techniques to suit any desired purpose. For example, an entire plant amino acid:glyoxylate aminotransferase gene sequence or portions thereof may be used as a probe capable of specifically hybridizing to coding sequences and messenger RNAs. To achieve specific hybridization under a variety of conditions, such probes include, e.g. sequences that are unique among plant amino acid:glyoxylate aminotransferase gene sequences and are at least 10 nucleotides in length, preferably at least 20 nucleotides in length, and most preferably at least 50 nucleotides in length. Such probes are used to amplify and analyze amino acid:glyoxylate aminotransferase gene sequences from a chosen organism via PCR. This technique is useful to isolate additional plant amino acid:glyoxylate aminotransferase gene sequences from a desired organism or as a diagnostic assay to determine the presence of amino acid:glyoxylate aminotransferase gene sequences in an organism. This technique also is used to detect the presence of altered amino acid:glyoxylate aminotransferase gene sequences associated with a particular condition of interest such as herbicide tolerance, poor health, etc.

Amino acid:glyoxylate aminotransferase specific hybridization probes also are used to map the location of these native genes in the genome of a chosen plant using standard techniques based on the selective hybridization of the probe to genomic sequences. These techniques include, but are not limited to, identification of DNA polymorphisms identified or contained within the probe sequence, and use of such polymorphisms to follow segregation of the gene relative to other markers of known map position in a mapping population derived from self fertilization of a hybrid of two polymorphic parental lines (see e.g. Helentjaris et al., Plant Mol. Biol. 5: 109 (1985); Sommer et al. Biotechniques 12:82 (1992); D'Ovidio et al., Plant Mol. Biol. 15: 169 (1990)). While any plant amino acid:glyoxylate aminotransferase gene sequence is contemplated to be useful as a probe for mapping amino acid:glyoxylate aminotransferase genes, preferred probes are those gene sequences from plant species more closely related to the chosen plant species, and most preferred probes are those gene sequences from the chosen plant species. Mapping of amino acid:glyoxylate aminotransferase genes in this manner is contemplated to be particularly useful for breeding purposes. For instance, by knowing the genetic map position of a mutant amino acid:glyoxylate aminotransferase gene that confers herbicide resistance, flanking DNA markers are identified from a reference genetic map (see, e.g., Helentjaris, Trends Genet. 3: 217 (1987)). During introgression of the herbicide resistance trait into a new breeding line, these markers are used to monitor the extent of linked flanking chromosomal DNA still present in the recurrent parent after each round of back-crossing.

Amino acid:glyoxylate aminotransferase specific hybridization probes also are used to quantify levels of amino acid:glyoxylate aminotransferase gene mRNA in a plant using standard techniques such as Northern blot analysis. This technique is useful as a diagnostic assay to detect altered levels of amino acid:glyoxylate aminotransferase gene expression that are associated with particular conditions such as enhanced tolerance to herbicides that target amino acid:glyoxylate aminotransferase genes.

II. Essentiality of Amino Acid:Glyoxylate Aminotransferase Genes in Plants Demonstrated by Antisense Inhibition

As shown in the examples below, the essentiality of amino acid:glyoxylate aminotransferase genes for normal plant growth and development is demonstrated by antisense inhibition of expression of the amino acid:glyoxylate aminotransferase gene in plants using the antisense validation system as described below. An antisense cDNA library is generated and cloned into an appropriate transformation vector. The thus created library of transformation vectors is used to produce a library of transgenic plants that express the random cDNA molecules in antisense orientation. The cellular functions of random cDNA clones are identified by screening mutant phenotypes in the transgenic plant pool. In the present invention, the library of transgenic plants is screened for plants with a seedling lethal phenotype. A cDNA clone responsible for a mutation is isolated by a simple cloning procedure involving polymerase chain reaction (PCR).

III. Recombinant Production of Plant Amino Acid:Glyoxylate Aminotransferase Enzymes and Uses Thereof

For recombinant production of a plant amino acid:glyoxylate aminotransferase enzyme in a host organism, a amino acid:glyoxylate aminotransferase coding sequence, preferably a plant coding sequence, is inserted into an expression cassette designed for the chosen host and introduced into the host where it is recombinantly produced. The choice of specific regulatory sequences such as promoter, signal sequence, 5′ and 3′ untranslated sequences, and enhancer appropriate for the chosen host is within the level of skill of the routineer in the art. The resultant molecule, containing the individual elements operably linked in proper reading frame, is inserted into a vector capable of being transformed into the host cell. Suitable expression vectors and methods for recombinant production of proteins are well known for host organisms such as E. coli, yeast, and insect cells (see, e.g., Luckow and Summers, Bio/Technol. 6: 47 (1988)). Specific examples include plasmids such as pBluescript (Stratagene, La Jolla, Calif.), pFLAG (International Biotechnologies, Inc., New Haven, Conn.), pTrcHis (Invitrogen, La Jolla, Calif.), and baculovirus expression vectors, e.g., those derived from the genome of Autographica californica nuclear polyhedrosis virus (AcMNPV). A preferred baculovirus/insect system is pV111392/Sf21 cells (Invitrogen, La Jolla, Calif.).

Recombinantly produced amino acid:glyoxylate aminotransferase enzymes is isolated and purified using a variety of standard techniques. The actual techniques used varies depending upon the host organism used, whether the enzyme is designed for secretion, and other such factors. Such techniques are well known to the skilled artisan (see, e.g. chapter 16 of Ausubel, F. et al., “Current Protocols in Molecular Biology”, pub. by John Wiley & Sons, Inc. (1994).

Recombinantly produced amino acid:glyoxylate aminotransferase enzymes are useful for a variety of purposes. For example, they are used in in vitro assays to screen known herbicidal chemicals, whose target has not been identified, to determine if they inhibit amino acid:glyoxylate aminotransferase enzymes. Such in vitro assays also are useful as screens to identify new chemicals that inhibit such enzymatic activity and that are therefore novel herbicide candidates. Alternatively, recombinantly produced amino acid:glyoxylate aminotransferase enzymes are used to further characterize their association with known inhibitors in order to rationally design new inhibitory herbicides as well as herbicide tolerant forms of the enzymes.

In Vitro Inhibitor Assay

An in vitro assay useful for identifying inhibitors of enzymes encoded by essential plant genes, such as amino acid:glyoxylate aminotransferase, comprises the steps of: a) reacting an enzyme having amino acid:glyoxylate aminotransferase activity and the substrate thereof in the presence of a suspected inhibitor of the enzyme's function; b) comparing the rate of enzymatic activities in the presence of the suspected inhibitor to the rate of enzymatic activities under the same conditions in the absence of the suspected inhibitor; and c) determining whether the suspected inhibitor inhibits the amino acid:glyoxylate aminotransferase enzymatic activity. The inhibitory effect on amino acid:glyoxylate aminotransferase activity is determined by a reduction or complete inhibition of product formation in the assay. In a preferred embodiment, such a determination is made by comparing, in the presence and absence of the candidate inhibitor, the amount of product formed in the in vitro assay using fluorescence or absorbance detection. In an in vitro assay for alanine:glyoxylate aminotransferase, the preferred substrates are L(+) alanine and glyoxylate.

In Vitro Inhibitor Assays: Discovery of Small Molecule Ligand that Interacts with the Gene Product of SEQ ID NO:1

Once a protein has been identified as a potential herbicide target, the next step is to develop an assay that allows screening large number of chemicals to determine which ones interact with the protein. Although it is straightforward to develop assays for proteins of known function, developing assays with proteins of unknown functions is more difficult.

This difficulty can be overcome by using technologies that can detect interactions between a protein and a compound without knowing the biological function of the protein. A short description of three methods is presented, including fluorescence correlation spectroscopy, surface-enhanced laser desorption/ionization, and biacore technologies.

Fluorescence Correlation Spectroscopy (FCS) theory was developed in 1972 but it is only in recent years that the technology to perform FCS became available (Madge et al. (1972) Phys. Rev. Lett., 29: 705-708; Maiti et al. (1997) Proc. Natl. Acad. Sci. USA, 94: 11753-11757). FCS measures the average diffusion rate of a fluorescent molecule within a small sample volume. The sample size can be as low as 10³ fluorescent molecules and the sample volume as low as the cytoplasm of a single bacterium. The diffusion rate is a function of the mass of the molecule and decreases as the mass increases. FCS can therefore be applied to protein-ligand interaction analysis by measuring the change in mass and therefore in diffusion rate of a molecule upon binding. In a typical experiment, the target to be analyzed is expressed as a recombinant protein with a sequence tag, such as a poly-histidine sequence, inserted at the N or C-terminus. The expression takes place in E. coli, yeast or insect cells. The protein is purified by chromatography. For example, the poly-histidine tag can be used to bind the expressed protein to a metal chelate column such as Ni2+ chelated on iminodiacetic acid agarose. The protein is then labeled with a fluorescent tag such as carboxytetramethylrhodamine or BODIPY® (Molecular Probes, Eugene, Oreg.). The protein is then exposed in solution to the potential ligand, and its diffusion rate is determined by FCS using instrumentation available from Carl Zeiss, Inc. (Thornwood, N.Y.). Ligand binding is determined by changes in the diffusion rate of the protein.

Surface-Enhanced Laser Desorption/Ionization (SELDI) was invented by Hutchens and Yip during the late 1980's (Hutchens and Yip (1993) Rapid Commun. Mass Spectrom. 7: 576-580). When coupled to a time-of-flight mass spectrometer (TOF), SELDI provides a mean to rapidly analyze molecules retained on a chip. It can be applied to ligand-protein interaction analysis by covalently binding the target protein on the chip and analyze by MS the small molecules that bind to this protein (Worrall et al. (1998) Anal. Biochem. 70: 750-756). In a typical experiment, the target to be analyzed is expressed as described for FCS. The purified protein is then used in the assay without further preparation. It is bound to the SELDI chip either by utilizing the poly-histidine tag or by other interaction such as ion exchange or hydrophobic interaction. The chip thus prepared is then exposed to the potential ligand via, for example, a delivery system capable to pipet the ligands in a sequential manner (autosampler). The chip is then submitted to washes of increasing stringency, for example a series of washes with buffer solutions containing an increasing ionic strength. After each wash, the bound material is analyzed by submitting the chip to SELDI-TOF. Ligands that specifically bind the target will be identified by the stringency of the wash needed to elute them.

Biacore relies on changes in the refractive index at the surface layer upon binding of a ligand to a protein immobilized on the layer. In this system, a collection of small ligands is injected sequentially in a 2-5 microlitre cell with the immobilized protein. Binding is detected by surface plasmon resonance (SPR) by recording laser light refracting from the surface. In general, the refractive index change for a given change of mass concentration at the surface layer, is practically the same for all proteins and peptides, allowing a single method to be applicable for any protein (Liedberg et al. (1983) Sensors Actuators 4: 299-304; Malmquist (1993) Nature, 361: 186-187). In a typical experiment, the target to be analyzed is expressed as described for FCS. The purified protein is then used in the assay without further preparation. It is bound to the Biacore chip either by utilizing the poly-histidine tag or by other interaction such as ion exchange or hydrophobic interaction. The chip thus prepared is then exposed to the potential ligand via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics on rate and off rate allows the discrimination between non-specific and specific interaction.

Also, an assay for small molecule ligands that interact with a polypeptide is an inhibitor assay. For example, such an inhibitor assay useful for identifying inhibitors of essential plant genes, such as plant amino acid:glyoxylate aminotransferase genes, comprises the steps of:

a) reacting a plant amino acid:glyoxylate aminotransferase enzyme and a substrate thereof in the presence of a suspected inhibitor of the enzyme's function;

b) comparing the rate of enzymatic activity in the presence of the suspected inhibitor to the rate of enzymatic activity under the same conditions in the absence of the suspected inhibitor; and

c) determining whether the suspected inhibitor inhibits the amino acid:glyoxylate aminotransferase enzyme.

For example, the inhibitory effect on plant amino acid:glyoxylate aminotransferase may be determined by a reduction or complete inhibition of amino acid:glyoxylate aminotransferase activity in the assay. Such a determination may be made by comparing, in the presence and absence of the candidate inhibitor, the amount of substrate used or intermediate or product made during the reaction.

IV. In Vivo Inhibitor Assay

In one embodiment, a suspected herbicide, for example identified by in vitro screening, is applied to plants at various concentrations. The suspected herbicide is preferably sprayed on the plants. After application of the suspected herbicide, its effect on the plants, for example death or suppression of growth is recorded.

In another embodiment, an in vivo screening assay for inhibitors of the amino acid:glyoxylate aminotransferase activity uses transgenic plants, plant tissue, plant seeds or plant cells capable of overexpressing a nucleotide sequence having amino acid:glyoxylate aminotransferase activity, wherein the amino acid:glyoxylate aminotransferase gene product is enzymatically active in the transgenic plants, plant tissue, plant seeds or plant cells. The nucleotide sequence is preferably derived from an eukaryote, such as a yeast, but is preferably derived from a plant. In a further preferred embodiment, the nucleotide sequence is identical or substantially identical to the nucleotide sequence set forth in SEQ ID NO:1, or encodes an enzyme having amino acid:glyoxylate aminotransferase activity, whose amino acid sequence is identical or substantially identical to the amino acid sequence set forth in SEQ ID NO:2. In another preferred embodiment, the nucleotide sequence is derived from a prokaryote.

A chemical is then applied to the transgenic plants, plant tissue, plant seeds or plant cells and to the isogenic non-transgenic plants, plant tissue, plant seeds or plant cells, and the growth or viability of the transgenic and non-transformed plants, plant tissue, plant seeds or plant cells are determined after application of the chemical and compared. Compounds capable of inhibiting the growth of the non-transgenic plants, but not affecting the growth of the transgenic plants are selected as specific inhibitors of amino acid:glyoxylate aminotransferase activity.

V. Herbicide Tolerant Plants

Development of tolerance can allow application of a herbicide to a crop where its use was previously precluded or limited (e.g. to pre-emergence use) due to sensitivity of the crop to the herbicide. For example, U.S. Pat. No. 4,761,373 to Anderson et al. is directed to plants resistant to various imidazolinone or sulfonamide herbicides. The resistance is conferred by an altered acetohydroxyacid synthase (AHAS) enzyme. U.S. Pat. No. 4,975,374 to Goodman et al. relates to plant cells and plants containing a gene encoding a mutant glutamine synthetase (GS) resistant to inhibition by herbicides that were known to inhibit GS, e.g. phosphinothricin and methionine sulfoximine. U.S. Pat. No. 5,013,659 to Bedbrook et al. is directed to plants expressing a mutant acetolactate synthase that renders the plants resistant to inhibition by sulfonylurea herbicides. U.S. Pat. No. 5,162,602 to Somers et al. discloses plants tolerant to inhibition by cyclohexanedione and aryloxyphenoxypropanoic acid herbicides. The tolerance is conferred by an altered acetyl coenzyme A carboxylase (ACCase).

The present invention is further directed to plants, plant tissue, plant seeds, and plant cells tolerant to herbicides that inhibit the naturally occurring amino acid:glyoxylate aminotransferase in these plants, wherein the tolerance is conferred by altered amino acid:glyoxylate aminotransferase enzyme activity. Altered amino acid:glyoxylate aminotransferase enzyme activity is conferred upon a plant according to the invention by increasing expression of wild-type herbicide-sensitive amino acid:glyoxylate aminotransferase enzyme by providing additional wild-type amino acid:glyoxylate aminotransferase genes to the plant, by expressing modified herbicide-tolerant amino acid:glyoxylate aminotransferase enzymes in the plant, or by a combination of these techniques. Representative plants include any plants to which these herbicides are applied for their normally intended purpose. Preferred are agronomically important crops such as cotton, soybean, oilseed rape, sugar beet, maize, rice, wheat, barley, oats, rye, sorghum, millet, turf, forage, turf grasses, and the like.

A. Increased Expression of Wild-Type Amino Acid:Glyoxylate Aminotransferase Enzymes

Achieving altered amino acid:glyoxylate aminotransferase enzyme activity through increased expression results in a level of a amino acid:glyoxylate aminotransferase enzyme in the plant cell at least sufficient to overcome growth inhibition caused by the herbicide. The level of expressed enzyme generally is at least two times, preferably at least five times, and more preferably at least ten times the natively expressed amount. Increased expression is conferred in a number of ways, e.g., providing multiple copies of a wild-type amino acid:glyoxylate aminotransferase gene; multiple occurrences of the coding sequence within the gene (i.e. gene amplification) or a mutation in the non-coding, regulatory sequence of the endogenous gene in the plant cell. Plants having such altered gene activity are obtained by direct selection in plants by methods known in the art (see, e.g. U.S. Pat. No. 5,162,602, and U.S. Pat. No. 4,761,373, and references cited therein). These plants also may be obtained by genetic engineering techniques known in the art. Increased expression of a herbicide-sensitive amino acid:glyoxylate aminotransferase gene also is accomplished by stably transforming a plant cell with a recombinant or chimeric DNA molecule comprising a promoter capable of driving expression of an associated structural gene in a plant cell operatively linked to a homologous or heterologous structural gene encoding the amino acid:glyoxylate aminotransferase enzyme.

B. Expression of Modified Herbicide-Tolerant Amino Acid:Glyoxylate Aminotransferase Enzymes

According to this embodiment, plants, plant tissue, plant seeds, or plant cells are stably transformed with a recombinant DNA molecule comprising a suitable promoter functional in plants operatively linked to a coding sequence encoding a herbicide tolerant form of a amino acid:glyoxylate aminotransferase enzyme. A herbicide tolerant form of the enzyme has at least one amino acid substitution, addition or deletion that confers tolerance to an amount of a herbicide effective to inhibit the unmodified, naturally occurring form of the amino acid:glyoxylate aminotransferase enzyme. The transgenic plants, plant tissue, plant seeds, or plant cells thus created are selected by conventional selection techniques, whereby herbicide tolerant lines are isolated, characterized, and developed. Below are described methods for obtaining genes that encode herbicide tolerant forms of amino acid:glyoxylate aminotransferase enzymes:

One strategy involves direct or indirect mutagenesis procedures on microbes. For instance, a genetically manipulatable microbe such as E. coli or S. cerevisiae may be subjected to random mutagenesis in vivo with mutagens such as UV light or ethyl or methyl methane sulfonate. Mutagenesis procedures are described, for example, in Miller, Experiments in Molecular Genetics, Cold Spring Harbor Laboratory, Cold Spring Harbor, N.Y. (1972); Davis et al., Advanced Bacterial Genetics, Cold Spring Harbor Laboratory, Cold Spring Harbor, N.Y. (1980); Sherman et al., Methods in Yeast Genetics, Cold Spring Harbor Laboratory, Cold Spring Harbor, N.Y. (1983); and U.S. Pat. No. 4,975,374. The microbe selected for mutagenesis contains a normal, inhibitor-sensitive amino acid:glyoxylate aminotransferase gene and is dependent upon the activity conferred by this gene. The mutagenized cells are grown in the presence of the inhibitor at concentrations that inhibit the unmodified gene. Colonies of the mutagenized microbe that grow better than the unmutagenized microbe in the presence of the inhibitor (i.e. exhibit resistance to the inhibitor) are selected for further analysis. Amino acid:glyoxylate aminotransferase genes from these colonies are isolated, either by cloning or by PCR amplification, and their sequences are elucidated. Sequences encoding altered gene products are then cloned back into the microbe to confirm their ability to confer inhibitor tolerance.

A method of obtaining mutant herbicide-tolerant alleles of a plant amino acid:glyoxylate aminotransferase gene involves direct selection in plants. For example, the effect of a mutagenized amino acid:glyoxylate aminotransferase gene on the growth inhibition of plants such as Arabidopsis, soybean, or maize is determined by plating seeds sterilized by art-recognized methods on plates on a simple minimal salts medium containing increasing concentrations of the inhibitor. Such concentrations are in the range of 0.001, 0.003, 0.01, 0.03, 0.1, 0.3, 1, 3, 10, 30, 110, 300, 1000 and 3000 parts per million (ppm). The lowest dose at which significant growth inhibition can be reproducibly detected is used for subsequent experiments.

Mutagenesis of plant material is utilized to increase the frequency at which resistant alleles occur in the selected population. Mutagenized seed material is derived from a variety of sources, including chemical or physical mutagenesis of seeds, or chemical or physical mutagenesis of pollen (Neuffer, In Maize for Biological Research Sheridan, ed. Univ. Press, Grand Forks, N. Dak., pp. 61-64 (1982)), which is then used to fertilize plants and the resulting M₁ mutant seeds collected. Typically for Arabidopsis M₂ seeds, which are progeny seeds of plants grown from seeds mutagenized with chemicals, such as ethyl methane sulfonate, or with physical agents, such as gamma rays or fast neutrons, are plated at densities of up to 10,000 seeds/plate (10 cm diameter) on minimal salts medium containing an appropriate concentration of inhibitor to select for tolerance. Seedlings that continue to grow and remain green 7-21 days after plating are transplanted to soil and grown to maturity and seed set. Progeny of these seeds are tested for tolerance to a amino acid:glyoxylate aminotransferase inhibitor. If the tolerance trait is dominant, plants whose seed segregate 3:1/resistant:sensitive are presumed to have been heterozygous for the resistance at the M₂ generation. Plants that give rise to all resistant seed are presumed to have been homozygous for the resistance at the M₂ generation. Such mutagenesis on intact seeds and screening of their M₂ progeny seed can also be carried out on other species, for instance soybean (see, e.g. U.S. Pat. No. 5,084,082). Alternatively, mutant seeds to be screened for herbicide tolerance are obtained as a result of fertilization with pollen mutagenized by chemical or physical means.

Confirmation that the genetic basis of the herbicide tolerance is a modified amino acid:glyoxylate aminotransferase gene is ascertained as exemplified below. First, alleles of the amino acid:glyoxylate aminotransferase gene from plants exhibiting resistance to the inhibitor are isolated using PCR with primers based either upon conserved regions in the Arabidopsis cDNA coding sequences shown in SEQ ID NO:1 or, more preferably, based upon the unaltered amino acid:glyoxylate aminotransferase gene sequence from the plant used to generate tolerant alleles. After sequencing the alleles to determine the presence of mutations in the coding sequence, the alleles are tested for their ability to confer tolerance to the inhibitor on plants into which the putative tolerance-conferring alleles have been transformed. These plants are Arabidopsis plants or any other plant whose growth is susceptible to the inhibitors. Second, the amino acid:glyoxylate aminotransferase genes are mapped relative to known restriction fragment length polymorphisms (RFLPs) (See, for example, Chang et al. Proc. Natl. Acad, Sci, USA 85: 6856-6860 (1988); Nam et al., Plant Cell 1: 699-705 (1989). The tolerance trait is independently mapped using the same markers. When tolerance is due to a mutation in that amino acid:glyoxylate aminotransferase gene, the tolerance trait maps to a position indistinguishable from the position of the amino acid:glyoxylate aminotransferase gene.

Another method of obtaining herbicide-tolerant alleles of a amino acid:glyoxylate aminotransferase gene is by selection in plant cell cultures. Explants of plant tissue, e.g. embryos, leaf disks, etc. or actively growing callus or suspension cultures of a plant of interest are grown on medium in the presence of increasing concentrations of a amino acid:glyoxylate aminotransferase inhibitor. Varying degrees of growth are recorded in different cultures. In certain cultures, fast-growing variant colonies arise that continue to grow even in the presence of normally inhibitory concentrations of inhibitor. The frequency with which such faster-growing variants occur can be increased by treatment with a chemical or physical mutagen before exposing the tissues or cells to the inhibitor. Putative tolerance-conferring alleles of the amino acid:glyoxylate aminotransferase gene are isolated and tested as described in the foregoing paragraphs. Those alleles identified as conferring herbicide tolerance may then be engineered for optimal expression and transformed into the plant. Alternatively, plants can be regenerated from the tissue or cell cultures containing these alleles.

Still another method involves mutagenesis of wild-type, herbicide sensitive plant amino acid:glyoxylate aminotransferase genes in bacteria or yeast, followed by culturing the microbe on medium that contains inhibitory concentrations of the inhibitor and then selecting those colonies that grow in the presence of the inhibitor. More specifically, a plant cDNA, such as the Arabidopsis cDNA encoding amino acid:glyoxylate aminotransferase (SEQ ID NO:1) is cloned into a microbe that otherwise lacks the selected gene's activity. The transformed microbe is then subjected to in vivo mutagenesis or to in vitro mutagenesis by any of several chemical or enzymatic methods known in the art, e.g. sodium bisulfite (Shortle et al., Methods Enzymol. 100:457-468 (1983); methoxylamine (Kadonaga et al., Nucleic Acids Res. 13:1733-1745 (1985); oligonucleotide-directed saturation mutagenesis (Hutchinson et al., Proc. Natl. Acad. Sci. USA, 83:710-714 (1986); or various polymerase misincorporation strategies (see, e.g. Shortle et al., Proc. Natl. Acad. Sci. USA, 79:1588-1592 (1982); Shiraishi et al., Gene 64:313-319 (1988); and Leung et al., Technique 1:11-15 (1989). Colonies that grow in the presence of normally inhibitory concentrations of inhibitor are picked and purified by repeated restreaking. Their plasmids are purified and tested for the ability to confer tolerance to the inhibitor by retransforming them into the microbe lacking amino acid:glyoxylate aminotransferase gene activity. The DNA sequences of cDNA inserts from plasmids that pass this test are then determined.

Herbicide resistant amino acid:glyoxylate aminotransferase proteins are also obtained using methods involving in vitro recombination, also called DNA shuffling. By DNA shuffling, mutations, preferably random mutations, are introduced into nucleotide sequences encoding amino acid:glyoxylate aminotransferase activity. DNA shuffling also leads to the recombination and rearrangement of sequences within a amino acid:glyoxylate aminotransferase gene or to recombination and exchange of sequences between two or more different of amino acid:glyoxylate aminotransferase genes. These methods allow for the production of millions of mutated amino acid:glyoxylate aminotransferase coding sequences. The mutated genes, or shuffled genes, are screened for desirable properties, e.g. improved tolerance to herbicides and for mutations that provide broad spectrum tolerance to the different classes of inhibitor chemistry. Such screens are well within the skills of a routineer in the art.

In a preferred embodiment, a mutagenized amino acid:glyoxylate aminotransferase gene is formed from at least one template amino acid:glyoxylate aminotransferase gene, wherein the template amino acid:glyoxylate aminotransferase gene has been cleaved into double-stranded random fragments of a desired size, and comprising the steps of adding to the resultant population of double-stranded random fragments one or more single or double-stranded oligonucleotides, wherein said oligonucleotides comprise an area of identity and an area of heterology to the double-stranded random fragments; denaturing the resultant mixture of double-stranded random fragments and oligonucleotides into single-stranded fragments; incubating the resultant population of single-stranded fragments with a polymerase under conditions which result in the annealing of said single-stranded fragments at said areas of identity to form pairs of annealed fragments, said areas of identity being sufficient for one member of a pair to prime replication of the other, thereby forming a mutagenized double-stranded polynucleotide; and repeating the second and third steps for at least two further cycles, wherein the resultant mixture in the second step of a further cycle includes the mutagenized double-stranded polynucleotide from the third step of the previous cycle, and the further cycle forms a further mutagenized double-stranded polynucleotide, wherein the mutagenized polynucleotide is a mutated amino acid:glyoxylate aminotransferase gene having enhanced tolerance to a herbicide which inhibits naturally occurring amino acid:glyoxylate aminotransferase activity. In a preferred embodiment, the concentration of a single species of double-stranded random fragment in the population of double-stranded random fragments is less than 1% by weight of the total DNA. In a further preferred embodiment, the template double-stranded polynucleotide comprises at least about 100 species of polynucleotides. In another preferred embodiment, the size of the double-stranded random fragments is from about 5 bp to 5 kb. In a further preferred embodiment, the fourth step of the method comprises repeating the second and the third steps for at least 10 cycles. Such method is described e.g. in Stemmer et al. (1994) Nature 370: 389-391, in U.S. Pat. No. 5,605,793, U.S. Pat. No. 5,811,238 and in Crameri et al. (1998) Nature 391: 288-291, as well as in WO 97/20078, and these references are incorporated herein by reference.

In another preferred embodiment, any combination of two or more different amino acid:glyoxylate aminotransferase genes are mutagenized in vitro by a staggered extension process (StEP), as described e.g. in Zhao et al. (1998) Nature Biotechnology 16: 258-261. The two or more amino acid:glyoxylate aminotransferase genes are used as template for PCR amplification with the extension cycles of the PCR reaction preferably carried out at a lower temperature than the optimal polymerization temperature of the polymerase. For example, when a thermostable polymerase with an optimal temperature of approximately 72° C. is used, the temperature for the extension reaction is desirably below 72° C., more desirably below 65° C., preferably below 60° C., more preferably the temperature for the extension reaction is 55° C. Additionally, the duration of the extension reaction of the PCR cycles is desirably shorter than usually carried out in the art, more desirably it is less than 30 seconds, preferably it is less than 15 seconds, more preferably the duration of the extension reaction is 5 seconds. Only a short DNA fragment is polymerized in each extension reaction, allowing template switch of the extension products between the starting DNA molecules after each cycle of denaturation and annealing, thereby generating diversity among the extension products. The optimal number of cycles in the PCR reaction depends on the length of the amino acid:glyoxylate aminotransferase genes to be mutagenized but desirably over 40 cycles, more desirably over 60 cycles, preferably over 80 cycles are used. Optimal extension conditions and the optimal number of PCR cycles for every combination of amino acid:glyoxylate aminotransferase genes are determined as described in using procedures well-known in the art. The other parameters for the PCR reaction are essentially the same as commonly used in the art. The primers for the amplification reaction are preferably designed to anneal to DNA sequences located outside of the amino acid:glyoxylate aminotransferase genes, e.g. to DNA sequences of a vector comprising the amino acid:glyoxylate aminotransferase genes, whereby the different amino acid:glyoxylate aminotransferase genes used in the PCR reaction are preferably comprised in separate vectors. The primers desirably anneal to sequences located less than 500 bp away from amino acid:glyoxylate aminotransferase sequences, preferably less than 200 bp away from the amino acid:glyoxylate aminotransferase sequences, more preferably less than 120 bp away from the amino acid:glyoxylate aminotransferase sequences. Preferably, the amino acid:glyoxylate aminotransferase sequences are surrounded by restriction sites, which are included in the DNA sequence amplified during the PCR reaction, thereby facilitating the cloning of the amplified products into a suitable vector. In another preferred embodiment, fragments of amino acid:glyoxylate aminotransferase genes having cohesive ends are produced as described in WO 98/05765. The cohesive ends are produced by ligating a first oligonucleotide corresponding to a part of a amino acid:glyoxylate aminotransferase gene to a second oligonucleotide not present in the gene or corresponding to a part of the gene not adjoining to the part of the gene corresponding to the first oligonucleotide, wherein the second oligonucleotide contains at least one ribonucleotide. A double-stranded DNA is produced using the first oligonucleotide as template and the second oligonucleotide as primer. The ribonucleotide is cleaved and removed. The nucleotide(s) located 5′ to the ribonucleotide is also removed, resulting in double-stranded fragments having cohesive ends. Such fragments are randomly reassembled by ligation to obtain novel combinations of gene sequences.

Any amino acid:glyoxylate aminotransferase gene or any combination of amino acid:glyoxylate aminotransferase genes, or homologs thereof, is used for in vitro recombination in the context of the present invention, for example, a amino acid:glyoxylate aminotransferase gene derived from a plant, such as, e.g. Arabidopsis thaliana, e.g. a amino acid:glyoxylate aminotransferase gene set forth in SEQ ID NO:1. Whole amino acid:glyoxylate aminotransferase genes or portions thereof are used in the context of the present invention. The library of mutated amino acid:glyoxylate aminotransferase genes obtained by the methods described above are cloned into appropriate expression vectors and the resulting vectors are transformed into an appropriate host, for example a plant cell, an algae like Chlamydomonas, a yeast or a bacteria. An appropriate host requires amino acid:glyoxylate aminotransferase gene product activity for growth. Host cells transformed with the vectors comprising the library of mutated amino acid:glyoxylate aminotransferase genes are cultured on medium that contains inhibitory concentrations of the inhibitor and those colonies that grow in the presence of the inhibitor are selected. Colonies that grow in the presence of normally inhibitory concentrations of inhibitor are picked and purified by repeated restreaking. Their plasmids are purified and the DNA sequences of cDNA inserts from plasmids that pass this test are then determined.

An assay for identifying a modified amino acid:glyoxylate aminotransferase gene that is tolerant to an inhibitor may be performed in the same manner as the assay to identify inhibitors of the amino acid:glyoxylate aminotransferase enzyme (Inhibitor Assay, above) with the following modifications: First, a mutant amino acid:glyoxylate aminotransferase enzyme is substituted in one of the reaction mixtures for the wild-type amino acid:glyoxylate aminotransferase enzyme of the inhibitor assay. Second, an inhibitor of wild-type enzyme is present in both reaction mixtures. Third, mutated activity (activity in the presence of inhibitor and mutated enzyme) and unmutated activity (activity in the presence of inhibitor and wild-type enzyme) are compared to determine whether a significant increase in enzymatic activity is observed in the mutated activity when compared to the unmutated activity. Mutated activity is any measure of activity of the mutated enzyme while in the presence of a suitable substrate and the inhibitor. Unmutated activity is any measure of activity of the wild-type enzyme while in the presence of a suitable substrate and the inhibitor. A significant increase is defined as an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, most preferably an increase by about 10-fold or greater.

In addition to being used to create herbicide-tolerant plants, genes encoding herbicide tolerant amino acid:glyoxylate aminotransferase enzymes also are used as selectable markers in plant cell transformation methods. For example, plants, plant tissue, plant seeds, or plant cells transformed with a transgene are transformed with a gene encoding an altered amino acid:glyoxylate aminotransferase enzyme capable of being expressed by the plant. The transformed cells are transferred to medium containing an amino acid:glyoxylate aminotransferase inhibitor in an amount sufficient to inhibit the survivability of plant cells not expressing the modified gene, wherein only the transformed cells will survive. The method is applicable to any plant cell capable of being transformed with a modified amino acid:glyoxylate aminotransferase enzyme-encoding gene, and can be used with any transgene of interest. Expression of the transgene and the inhibitor-tolerant amino acid:glyoxylate aminotransferase gene can be driven by the same promoter functional in plant cells, or by separate promoters.

In yet another embodiment, herbicide-resistant amino acid:glyoxylate aminotransferase proteins are produced using the incremental truncation for the creation of hybrid enzymes (ITCHY), as described in Ostermeier et al. (1999) Nature Biotechnology 17:1205-1209), and this reference is incorporated herein by reference.

VI. Plant Transformation Technology

A wild-type or herbicide-tolerant form of the amino acid:glyoxylate aminotransferase gene can be incorporated in plant or bacterial cells using conventional recombinant DNA technology. Generally, this involves inserting a DNA molecule encoding the amino acid:glyoxylate aminotransferase enzyme into an expression system to which the DNA molecule is heterologous (i.e., not normally present) using standard cloning procedures known in the art. The vector contains the necessary elements for the transcription and translation of the inserted protein-coding sequences in a host cell containing the vector. A large number of vector systems known in the art can be used, such as plasmids, bacteriophage viruses and other modified viruses. The components of the expression system optionally are modified to increase expression. For example, truncated sequences, nucleotide substitutions or other modifications optionally are employed. Expression systems known in the art are used to transform virtually any crop plant cell under suitable conditions. Transformed cells are regenerated into whole plants such that the chosen form of the amino acid:glyoxylate aminotransferase gene confers herbicide tolerance in the transgenic plants.

A. Requirements for Construction of Plant Expression Cassettes

Gene sequences intended for expression in transgenic plants are first operably linked to a suitable promoter expressible in plants. Such expression cassettes optionally comprise further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes are easily transferred to the plant transformation vectors described infra. The following is a description of various components of typical expression cassettes.

1. Promoters

The selection of the promoter used determines the spatial and temporal expression pattern of the transgene in the transgenic plant. Selected promoters will express transgenes in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the gene under various inducing conditions. Promoters vary in their strength, i.e., ability to promote transcription. Depending upon the host cell system utilized, any one of a number of suitable promoters known in the art can be used. For example, for constitutive expression, the CaMV 35S promoter, the rice actin promoter, or the ubiquitin promoter may be used. For regulatable expression, the chemically inducible PR-1 promoter from tobacco or Arabidopsis may be used (see, e.g., U.S. Pat. No. 5,689,044).

2. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tml terminator, the nopaline synthase terminator and the pea rbcS E9 terminator. These can be used in both monocotyledons and dicotyledons.

3. Sequences for the Enhancement or Regulation of Expression

Numerous sequences are known to enhance gene expression from within the transcriptional unit and these sequences can be used in conjunction with the genes of this invention to increase their expression in transgenic plants. For example, various intron sequences such as introns of the maize AdhI gene have been shown to enhance expression, particularly in monocotyledonous cells. In addition, a number of non-translated leader sequences derived from viruses also are known to enhance expression, and these are particularly effective in dicotyledonous cells.

4. Coding Sequence Optimization

The coding sequence of the selected gene optionally is genetically engineered by altering the coding sequence for optimal expression in the crop species of interest. Methods for modifying coding sequences to achieve optimal expression in a particular crop species are well known (see, e.g. Perlak et al., Proc. Natl. Acad. Sci. USA 88: 3324 (1991); and Koziel et al., Bio/technol. 11: 194 (1993); Fennoy and Bailey-Serres. Nucl. Acids Res. 21: 5294-5300 (1993). Methods for modifying coding sequences by taking into account codon usage in plant genes and in higher plants, green algae, and cyanobacteria are well known (see table 4 in: Murray et al. Nucl. Acids Res. 17: 477-498 (1989); Campbell and Gowri Plant Physiol. 92: 1-11(1990).

5. Targeting of the Gene Product Within the Cell

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins which is cleaved during chloroplast import to yield the mature protein (e.g. Comai et al. J. Biol. Chem. 263: 15104-15109 (1988)). Other gene products are localized to other organelles such as the mitochondrion and the peroxisome (e.g. Unger et al. Plant Molec. Biol. 13: 411-418 (1989)). The cDNAs encoding these products are manipulated to effect the targeting of heterologous gene products to these organelles. In addition, sequences have been characterized which cause the targeting of gene products to other cell compartments. Amino terminal sequences are responsible for targeting to the ER, the apoplast, and extracellular secretion from aleurone cells (Koehler & Ho, Plant Cell 2: 769-783 (1990)). Additionally, amino terminal sequences in conjunction with carboxy terminal sequences are responsible for vacuolar targeting of gene products (Shinshi et al. Plant Molec. Biol. 14: 357-368 (1990)). By the fusion of the appropriate targeting sequences described above to transgene sequences of interest one skilled in the art is able to direct the transgene product to any organelle or cell compartment.

B. Construction of Plant Transformation Vectors

Numerous transformation vectors available for plant transformation are known to those of ordinary skill in the plant transformation arts, and the genes pertinent to this invention are used in conjunction with any such vectors. The selection of vector will depend upon the preferred transformation technique and the target species for transformation. For certain target species, different antibiotic or herbicide selection markers may be preferred. Selection markers used routinely in transformation include the nptII gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), and the dhfr gene, which confers resistance to methotrexate (Bourouis et al., EMBO J. 2(7): 1099-1104 (1983)), and the EPSPS gene, which confers resistance to glyphosate (U.S. Pat. Nos. 4,940,935 and 5,188,642).

1. Vectors Suitable for Agrobacterium Transformation

Many vectors are available for transformation using Agrobacterium tumefaciens. These typically carry at least one T-DNA border sequence and include vectors such as pBIN19 (Bevan, Nucl. Acids Res. (1984)). Typical vectors suitable for Agrobacterium transformation include the binary vectors pCIB200 and pCIB2001, as well as the binary vector pCIB10 and hygromycin selection derivatives thereof. (See, for example, U.S. Pat. No. 5,639,949).

2. Vectors Suitable for non-Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Typical vectors suitable for non-Agrobacterium transformation include pCIB3064, pSOG19, and pSOG35. (See, for example, U.S. Pat. No. 5,639,949).

C. Transformation Techniques

Once the coding sequence of interest has been cloned into an expression system, it is transformed into a plant cell. Methods for transformation and regeneration of plants are well known in the art. For example, Ti plasmid vectors have been utilized for the delivery of foreign DNA, as well as direct DNA uptake, liposomes, electroporation, micro-injection, and microprojectiles. In addition, bacteria from the genus Agrobacterium can be utilized to transform plant cells.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. In each case the transformed cells are regenerated to whole plants using standard techniques known in the art.

Transformation of most monocotyledon species has now also become routine. Preferred techniques include direct gene transfer into protoplasts using PEG or electroporation techniques, particle bombardment into callus tissue, as well as Agrobacterium-mediated transformation.

D. Plastid Transformation

In another preferred embodiment, a nucleotide sequence encoding a polypeptide having amino acid:glyoxylate aminotransferase activity is directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit expression levels that can readily exceed 10% of the total soluble plant protein. In a preferred embodiment, the nucleotide sequence is inserted into a plastid targeting vector and transformed into the plastid genome of a desired plant host. Plants homoplasmic for plastid genomes containing the nucleotide sequence are obtained, and are preferentially capable of high expression of the nucleotide sequence.

Plastid transformation technology is for example extensively described in U.S. Pat. Nos. 5,451,513, 5,545,817, 5,545,818, and 5,877,462 in PCT application no. WO 95/16783 and WO 97/32977, and in McBride et al. (1994) Proc. Natl. Acad. Sci. USA 91, 7301-7305, all incorporated herein by reference in their entirety. The basic technique for plastid transformation involves introducing regions of cloned plastid DNA flanking a selectable marker together with the nucleotide sequence into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab, Z., and

Maliga, P. (1993) Proc. Natl. Acad. Sci. USA 90, 913-917). Other selectable markers useful for plastid transformation are known in the art and encompassed within the scope of the invention.

VII. Breeding

The wild-type or altered form of a amino acid:glyoxylate aminotransferase gene of the present invention is utilized to confer herbicide tolerance to a wide variety of plant cells, including those of gymnosperms, monocots, and dicots. Although the gene can be inserted into any plant cell falling within these broad classes, it is particularly useful in crop plant cells, such as rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

The high-level expression of a wild-type amino acid:glyoxylate aminotransferase gene and/or the expression of herbicide-tolerant forms of a amino acid:glyoxylate aminotransferase gene conferring herbicide tolerance in plants, in combination with other characteristics important for production and quality, is incorporated into plant lines through breeding approaches and techniques known in the art.

Where a herbicide tolerant amino acid:glyoxylate aminotransferase gene allele is obtained by direct selection in a crop plant or plant cell culture from which a crop plant can be regenerated, it is moved into commercial varieties using traditional breeding techniques to develop a herbicide tolerant crop without the need for genetically engineering the allele and transforming it into the plant.

The invention will be further described by reference to the following detailed examples. These examples are provided for purposes of illustration only, and are not intended to be limiting unless otherwise specified.

### EXAMPLES

Standard recombinant DNA and molecular cloning techniques used here are well known in the art and are described by Sambrook, et al., Molecular Cloning, eds., Cold Spring Harbor Laboratory Press, Cold Spring Harbor, N.Y. (1989) and by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987). Arabidopsis thaliana (L.) Heynh (WS-O ecotype) is used in the following examples, and plants are grown in growth chambers with a 16 hour light/8 hour dark cycle at 22C.

### Example 1

### Construction of a Plant Antisense Expression Vector pNB96

A plant antisense expression vector, pNB96, is constructed as follows. The EcoR I/Sma I fragment of the TEV leader sequence in pTRL2 (Ling et al. (1991) Biotechnology, 9: 752-758) is replaced by a synthetic oligonucleotide sequence containing Eco RI/Not I sites. The inserted sequence is 5′-AAT TGC GGC CGC AAA GAA TTC-3′ (oligonucleotide A: SEQ ID NO:5). The Hind III fragment containing the 35S promoter-EcoR I/Not I sites-35S terminator is, then, subcloned into the Hind III-digested pARK5 (Saito et al. (1992) Plant Cell Reports, 11: 219-224), after filling the EcoR I and Not I sites in pARK5. The resulting antisense expression vector is named pNB96.

The vector pNB96 contains the Not I and EcoR I sites downstream from the dual 35S promoter for cloning of the cDNA molecules in the antisense direction, and the 35S terminator is downstream of the EcoR I cloning site. The npt II and bar gene encodes kanamycin and basta resistance, respectively. Expression of the npt II gene is driven by the nos promoter and the nos terminator is downstream of the npt II gene. Expression of the bar gene is driven by the 35S promoter and the nos terminator is downstream of the bar gene. The positions of the primer sequences used for isolating inserted cDNAs by PCR are indicated by P1 P4, respectively. The nucleotide sequences corresponding to the primers are the following: primer P1 (5′-TTC GCA AGA CCC TTC CTC TA-3′: SEQ ID NO:6) and primer P4 (5′-CTT ATC TGG GAA CTA CTC-3′: SEQ ID NO:7).

### Example 2

### Construction of the Antisense cDNA Library of Arabidopsis thaliana

An antisense cDNA library of Arabidopsis thaliana in this vector is generated using RNA from leaf tissue of Arabidopsis. Total RNA isolated from leaf tissues of Arabidopsis thaliana is used for preparation of poly (A)⁺ RNA. Double-stranded cDNA is constructed from 5 μg of poly (A)⁺ RNA with the Time Saver cDNA synthesis Kit (Pharmacia) using Not I-dT₁₈ as a primer. The cDNA is inserted into the antisense vector pNB96 after ligating an EcoR I adapter and digesting with Not I.

The antisense cDNA library is first established in E. coli. The primary cDNA library established in the E. coli XL-1 Blue cells (Bullock et al. (1987) BioTechniques, 5: 376-378) contains 2×10⁵ recombinant clones. The sizes of the antisense cDNA inserts estimated from 50 random clones are between 150 and 3400 bp. The plasmid DNA prepared from this library is then introduced into the Agrobacterium strain AGL1 (Lazo et al. (1991) Bio/Technology, 9: 963-967). The primary titer of the Arabidopsis antisense cDNA library established in Agrobacterium is approximately 2×10⁵.

### Example 3

### Generation of the Random Antisense Transgenic Plant Pool

The antisense cDNA library established in Agrobacterium is used to generate a transgenic plant library of Arabidopsis. Arabidopsis thaliana (L.) Heynh (WS-O ecotype) is used for transformation as follows. The Agrobacterium culture is grown at 28° C. to a density of 0.8-1.0 (OD₆₀₀) and harvested by centrifugation at 5,000 rpm for 10 min at 25° C. The bacterial pellet is resuspended in IM (Infiltration Media: 1×MS salt, 1×B5 vitamin, 5% sucrose, 0.005% Silwet L-77) to a final OD₆₀₀ of 2.0˜2.4. Four week-old plants are immersed in Agrobacterium suspension in a vacuum chamber and put under vacuum (15 in. Hg) for 10 min. After infiltration, plants are kept covered with a polyethylene foil for 24 h. Thereafter plants are grown to maturity and seeds (T1) are harvested in bulk. Transgenic plants are selected by soaking the seeds in 0.1% basta solution and then growing the plants in soil. The transformation efficiency is 0.5%.

### Example 4

### Identification of the Lethal Mutant Line 35-191 at the T1 Generation

The basta-sensitive and non-transgenic seedlings do not grow beyond the seedling stage, whereas transgenic, basta-resistant seedlings grow beyond the seedling stage. Since antisense effect is primarily dominant, some antisense mutants may be found in the T1 generation. Among the antisense transgenic lines that survived beyond the seedling stage, approximately 1% of the T1 transgenic plants show lethality or sterility at the T1 generation. Among these lines, a line designated 35-191 shows severe bleaching of the rosette leaves and cauline leaves. Later, the floral part is degenerated and thus the plants do not set seeds. Finally, the plants die much earlier than wild type plants.

### Example 5

### Cloning of the cDNA Insert From the 35-191 Line by Polymerase Chain Reaction

One of the advantages of the antisense validation system described herein is its simplicity in cloning the inserted cDNA responsible for a mutant phenotype, since the cDNA clones in these lines can be isolated by a simple PCR using the vector sequences surrounding the inserted cDNA as primers. cDNA clones from the 35-191 mutant lines are isolated by polymerase chain reaction (PCR). For the PCR reaction, 50 μl of the reaction mixture is prepared to contain 25 μl of the quick start PCR mix (TaKaRa), 100 ng of genomic DNA and 200 ng each of the primers. PCR is performed for 35 cycles (30 sec at 94° C., 30 sec at 52° C., and 1 min at 68° C.) with the primer set P1 (5′-TTC GCA AGA CCC TTC CTC TA-3′: SEQ ID NO:6) and primer P4 (5′-CTT ATC TGG GAA CTA CTC-3′: SEQ ID NO:7). The size of the cloned cDNA insert from line 35-191 is 1.0 kb.

### Example 6

### Determination of the Partial Sequence of the cDNA Clone Isolated from the 35-191 Line

The PCR product is cloned into pGEM-T Easy vector (Promega Biotech). The partial sequences of the cDNA clone are determined from both ends. Sequencing is performed with double-stranded DNA by the dideoxy chain termination method using a ThermoSequenase kit (Amersham). SP6 and T7 primers are used to determine the sequences from the 5′ and 3′ ends of the clone, respectively. The sequence is then compared against the sequences in the databases by the Blast program. The nucleotide sequence corresponding to the 5′ portion of the cDNA clone (SEQ ID NO:3), and the nucleotide sequence corresponding to the 3′ portion of the cDNA clone (SEQ ID NO:4), are identical to the 5′ and 3′ ends of the Arabidopsis alanine:glyoxylate aminotransferase mRNA (Genbank accession #: AF063901, SEQ ID NO:1) except one base pair. The difference is likely due to the differences in the ecotypes of plants used in our experiment (WS-O) from that in the Genbank database (Col-O).

### Example 7

### Retransformation

To confirm that antisense expression of the insert cDNAs isolated from mutant line 35-191 is responsible for the phenotypes, the cDNA fragment isolated from the mutant line is recloned and introduced into wild type plants. The cDNA clone is digested with Not I and Eco RI, and cloned into pNB96 vector for plant transformation. When phenotypes of these transgenic plants are examined at the T1 generation, 8 plants out of 20 plants that survive beyond the seedling stage after basta selection show the same mutant phenotype as the original mutation, although there is some variability in severity of mutant phenotypes. This result confirms that antisense expression of the cDNA clone is responsible for the mutant phenotype and that the amino acid:glyoxylate aminotransferase is an essential gene.

### Example 8

### Expression of Recombinant Amino Acid:Glyoxylate Aminotransferase Protein in E. coli

The coding region of the protein, corresponding to the cDNA clone SEQ ID NO:1, is subcloned into an appropriate expression vector, and transformed into E. coli using the manufacturer's conditions. Specific examples include plasmids such as pBluescript (Stratagene, La Jolla, Calif.), pFLAG (International Biotechnologies, Inc., New Haven, Conn.), and pTrcHis (Invitrogen, La Jolla, Calif.). E. coli is cultured, and expression of amino acid:glyoxylate aminotransferase activity is confirmed. Protein conferring amino acid:glyoxylate aminotransferase activity is isolated using standard techniques.

### Example 9

### In vitro Recombination of Amino Acid:Glyoxylate Aminotransferase Genes by DNA Shuffling

The nucleotide sequence of SEQ ID NO:1 is amplified by PCR. The resulting DNA fragment is digested by DNaseI treatment essentially as described (Stemmer et al. (1994) PNAS 91: 10747-10751) and the PCR primers are removed from the reaction mixture. A PCR reaction is carried out without primers and is followed by a PCR reaction with the primers, both as described (Stemmer et al. (1994) PNAS 91: 10747-10751). The resulting DNA fragments are cloned into pTRC99a (Pharmacia, Cat no: 27-5007-01) for use in bacteria, and transformed into a bacterial strain deficient in amino acid:glyoxylate aminotransferase activity by electroporation using the BioRad Gene Pulser and the manufacturer's conditions. The transformed bacteria are grown on medium that contains inhibitory concentrations of an inhibitor of amino acid:glyoxylate aminotransferase activity and those colonies that grow in the presence of the inhibitor are selected. Colonies that grow in the presence of normally inhibitory concentrations of inhibitor are picked and purified by repeated restreaking. Their plasmids are purified and the DNA sequences of cDNA inserts from plasmids that pass this test are then determined. Alternatively, the DNA fragments are cloned into expression vectors for transient or stable transformation into plant cells, which are screened for differential survival and/or growth in the presence of an inhibitor of amino acid:glyoxylate aminotransferase activity. In a similar reaction, PCR-amplified DNA fragments comprising the Arabidopsis amino acid:glyoxylate aminotransferase gene encoding the protein and PCR-amplified DNA fragments derived from or comprising another amino acid:glyoxylate aminotransferase gene are recombined in vitro and resulting variants with improved tolerance to the inhibitor are recovered as described above.

### Example 10

### In vitro Recombination of Amino Acid:Glyoxylate Aminotransferase Genes by Staggered Extension Process

The Arabidopsis amino acid:glyoxylate aminotransferase gene and another amino acid:glyoxylate aminotransferase gene, or homologs thereof, or fragments thereof, are each cloned into the polylinker of a pBluescript vector. A PCR reaction is carried out essentially as described (Zhao et al. (1998) Nature Biotechnology 16: 258-261) using the “reverse primer” and the “M13-20 primer” (Stratagene Catalog). Amplified PCR fragments are digested with appropriate restriction enzymes and cloned into pTRC99a and mutated amino acid:glyoxylate aminotransferase genes are screened as described in Example 9.

### Example 11

### In Vitro Binding Assays

Recombinant amino acid:glyoxylate aminotransferase protein is obtained, for example, according to Example 8. The protein is immobilized on chips appropriate for ligand binding assays using techniques that are well known in the art. The protein immobilized on the chip is exposed to sample compound in solution according to methods well know in the art. While the sample compound is in contact with the immobilized protein measurements capable of detecting protein-ligand interactions are conducted. Examples of such measurements are SELDI, biacore and FCS, described above. Compounds found to bind the protein are readily discovered in this fashion and are subjected to further characterization.

### Example 12

### Alanine:Glyoxylate Aminotransferase Activity Assay

The alanine:glyoxylate aminotransferase activity assay is derived from Stintjes et al. (1992) Anal. Biochem. 206, 334-343. The reaction volumes are preferably the ones described below, but can be varied depending on the experimental requirements. 0.01-1.0×10⁻³ unit of an enzyme having alanine:glyoxylate aminotransferase activity (one [symbol] of activity is defined as the amount of enzyme required to produce 1 μmol/min of produ [symbol] and 0.1-10 mM, but preferably 2 mM, L(+)alanine and 0.1-10 mM, but preferably 0.75 mM, glyoxylate are mixed in a final volume of 10 μL 10 mM Tris-HCl (pH 7.0-9.0, but preferably 8.5) and 1-20 μM, but preferably 10 μM pyridoxal 5′-phosphate. The production of pyruvate is determined preferably according to Stintjes et al. (1992) Anal. Biochem. 206, 334-343 by adding 5 μL of 20 mM o-phenylenediamine in 0.6 M hydrochloric acid. Fluorescence intensity is measured for the solution with an excitation wavelength of 410±10 nm and an emission wavelength of 535±10 nm. Alternatively, the absorbance of the solution may be measured with a wavelength of 410±10 nm.

Alternatively, pyruvate formation is quantitated by a coupled reaction procedure. In this case, 0.5 units of lactate dehydrogenase and 0.2 mM NAD [symbol] are added and the fluorescence intensity of the solution is measured with an excitation wavelength of 340±10 nm and an emission wavelength of 410±10 nm. Alternatively, the absorbance of the solution may be measured at 340 nm.

Various modifications of the invention described herein will become apparent to those skilled in the art. Such modifications are intended to fall within the scope of the appended claims.

What is claimed is:

1. A method of selecting a herbicidal compound, comprising:
(a) combining a polypeptide comprising an amino acid sequence at least 90% identical to SEQ ID NO:2 and a compound to be tested for the ability to bind to said polypeptide under conditions conducive to binding;
(b) selecting a compound identified in step (a) that binds to said polypeptide;
(c) applying a compound selected in step (b) to a plant to test for herbicidal activity; and
(d) selecting a compound identified in step (c) that has herbicidal activity.

2. The method of claim 1, wherein said polypeptide has amino acid:glyoxylate aminotransferase activity.

3. The method of claim 1, wherein said polypeptide comprises an amino acid sequence at least 95% identical to SEQ ID NO:2.

4. The method of claim 1, wherein said polypeptide comprises SEQ ID NO:2.

5. An isolated polypeptide having amino acid:glyoxylate aminotransferase activity, wherein said polypeptide comprises an amino acid sequence at least 90% identical to SEQ ID NO:2.

6. The isolated polypeptide of claim 5, wherein said polypeptide comprises an amino acid sequence at least 95% identical to SEQ ID NO:2.

7. The isolated polypeptide of claim 6, wherein said polypeptide comprises SEQ ID NO:2.

8. An isolated nucleic acid molecule comprising a nucleotide sequence encoding a polypeptide having amino acid:glyoxylate aminotransferase activity, wherein said polypeptide comprises an amino acid sequence at least 90% identical to SEQ ID NO:2.

9. The isolated nucleic acid molecule of claim 8, wherein said polypeptide comprises an amino acid sequence at least 95% identical to SEQ ID NO:2.

10. The isolated nucleic acid molecule of claim 9, wherein said polypeptide comprises SEQ ID NO:2.

11. An expression cassette comprising a promoter operatively linked to the nucleic acid molecule of claim 8.

12. A recombinant vector comprising the expression cassette of claim 11.

13. A transgenic host cell comprising the expression cassette of claim 11.

14. The transgenic host cell of claim 13, wherein said host cell is selected from the group consisting of an insect cell, a yeast cell, a prokaryotic cell, and a plant cell.

15. A transgenic plant cell comprising the expression cassette of claim 11.

16. A transgenic plant or seed comprising the transgenic plant cell of claim 15.


SEQUENCE LISTING (7 sequences)

SEQ ID NO: 1 - DNA, 1452 bp, Arabidopsis thaliana
  CDS (71)..(1276)
ccacgcgtccgctcaggagcttccgccttattcagagtgtagaggaggatccaaaagaaaagaggaaaaaatggactatatgtatggaccagggagacaccatctgtttgtaccaggaccagtgaacataccggaaccggtaatccgggcgatgaaccggaacaacgaggattaccggtcaccagccattccggcgcttacgaaaacattgttggaggatgttaagaagatattcaagaccacatcagggacaccttttctgtttcccacgaccgggactggtgcttgggagagtgccttgaccaacacgttatctcctggagacaggattgtttcgtttctgattggacaatttagcttgctctggattgaccagcagaagaggcttaatttcaatgttgatgtggttgagagtgattggggacaaggtgctaatctccaagtcttggcctcaaagctctcacaagacgagaatcataccatcaaagccatttgcattgtccacaacgagaccgcgaccggagttaccaatgacatctctgctgtccgcacactcctcgatcactacaagcatccggctttgctgctagtggacggtgtttcgtccatctgcgcgcttgatttccgaatggatgagtggggagtggacgtggccttgactgggtctcagaaagccttatctcttccaacaggacttggtattgtctgcgccagtcctaaagctttggaagctaccaaaacttctaaatctctcaaagtattctttgactggaatgactaccttaagttttacaagctaggaacctattggccatacacaccttccattcaacttctctacggtcttagagctgctcttgatcttatctttgaggaaggacttgagaacatcatcgcccgccatgctcgtttgggaaaggccaccaggcttgcggtggaagcatgggggctgaaaaactgcacacagaaggaggaatggataagtaacacagtgacagcagttatggtgcctccgcatatagacggttcggagattgtgagaagggcatggcagaggtacaacttaagtcttggtcttggtctcaacaaagtggctggaaaggttttcagaattggacatctaggaaatgtgaatgagttgcaacttctcgggtgtcttgcgggagtggagatgatactgaaggatgttggatacccagttgtaatgggaagtggagttgcagctgcctctacttatcttcagcaccacattcctctcattccctctagaatctaatccatgtggtccttctctttctttctcttcctctcaatgtaaacaaactctcatgttttctcttttcttcatctatatttttactacattatctatgtttttatttgctatctccaacaaacttattgaacaaaagatcaaatatgtttattactcactaaaaaaaaaaaaaaaaa

SEQ ID NO: 2 - protein, 401 aa, Arabidopsis thaliana [sequence omitted]

SEQ ID NO: 3 - DNA, 145 bp, Arabidopsis thaliana
  misc_feature (1)..(145): 5′ portion of the partial cDNA nucleotide sequence
ctcaaagctctcacaagacgagaatcataccatcaaagccatttgcattgtccacaacgagaccgcgaccggagttaccaatgacatctctgctgtccgcacactcctcgatcactacaagcatccggctttgctgctagtggac

SEQ ID NO: 4 - DNA, 204 bp, Arabidopsis thaliana
  misc_feature (1)..(204): 3′ portion of the partial cDNA nucleotide sequence
gcgggagtggagatgatactgaaggatgttggatacccagttgtaatgggaagtggagttgcatctgcctctacttatcttcagcaccacattcctctcattccctctagaatctaatccatgtggtccttctctttctttctcttcctctcaatgtaaacaaactctcatgttttctcttttcttcatctatatttttactac

SEQ ID NO: 5 - DNA, 21 bp, Artificial Sequence
  Description of Artificial Sequence oligonucleotide
aattgcggccgcaaagaattc

SEQ ID NO: 6 - DNA, 20 bp, Artificial Sequence
  Description of Artificial Sequence oligonucleotide
ttcgcaagacccttcctcta

SEQ ID NO: 7 - DNA, 18 bp, Artificial Sequence
  Description of Artificial Sequence oligonucleotide
cttatctgggaactactc